SEQUENCE LISTING (9 sequences)

SEQ ID NO: 1 - RNA, 8 nt, Artificial Sequence
  Synthetic
uggugcaa

SEQ ID NO: 2 - RNA, 5 nt, Artificial Sequence
  Synthetic
cgccu

SEQ ID NO: 3 - RNA, 5 nt, Artificial Sequence
  Synthetic
gauuu

SEQ ID NO: 4 - RNA, 5 nt, Artificial Sequence
  Synthetic
cuaaa

SEQ ID NO: 5 - DNA, 11 bp, Artificial Sequence
  Synthetic
tggcnnagtgg

SEQ ID NO: 6 - DNA, 11 bp, Artificial Sequence
  Synthetic
ggttcganncc

SEQ ID NO: 7 - DNA, 11 bp, Artificial Sequence
  Synthetic
ggttcganncc

SEQ ID NO: 8 - DNA, 11 bp, Artificial Sequence
  Synthetic
tatauuucgcg

SEQ ID NO: 9 - DNA, 12 bp, Artificial Sequence
  Synthetic
uuucgccctata

(19) United States
(12) Patent Application Publication
(10) Pub. No.: US 2011/0213006 A1
(43) Pub. Date: Sep. 1, 2011

(54) Compositions and Methods for Treatment of Uncontrolled Cell Growth

(75) Inventor: Syed K. Hasan, Lowell, MA (US)

(73) Assignee: IMMUNOTREX CORPORATION, Lowell, MA (US)

(21) Appl. No.: 12/596,753

(22) PCT Filed: Apr. 21, 2008
(86) PCT No.: PCT/US2008/061038

§ 371 Date: Jul. 1, 2010

Related U.S. Application Data
(60) Provisional application No. 60/913,201, filed on Apr. 20, 2007.

(51) Int. Cl. A61K 31/7088; C07H 21/02; C07H 21/04; A61P 35/00; A61P 35/02; A61K 31/7105

(52) U.S. Cl. 514/44 A; 536/24.5

(57) ABSTRACT

Compositions and methods are provided for the treatment of cancer and other diseases of uncontrolled cell growth. Inhibitors of t-RNA and 28s rRNA are provided as are non-functional amino acid residues for charging of t-RNA molecules. Therapeutic application of the above inhibitors is also provided for the treatment of cancer.

### TECHNICAL FIELD

The present invention relates to compositions and methods for inhibition of cell growth. More particularly, the present invention relates to cancer therapies based on inhibition of t-RNA and 28s rRNA activity in cancer cells.

### BACKGROUND OF THE INVENTION

Cancer research directed at the inhibition of t-RNA's in eukaryotic cells took a back seat in the late 1970s with the discovery of specific transcription factors and the potential for modifying signal transduction in cancer cells based on these transcription factors.

Discovery of a stable and specific inhibitors of tumor cell transcription and translation has as yet to be successful. The inability of researchers to develop useful inhibitors based on targeted transcription factors has partly been due to the complex nature of gene transcription, including chromosome packaging in the cell, i.e., histone/chromatin, complex nature of signal transduction in any give cell type, as well as the duplicative and complex nature of how various DNA/RNA binding proteins influence cell growth.

However, it is also known from these studies that inhibition of protein production in a target cell will lead to a cell's death, especially where the inhibition of protein production is of the so called “house keeping proteins.”

Therefore, there is a need in the art to identify new technologies involved with targeting and inhibiting protein production in cancer cells and for using these new technologies in new therapeutics for treating various cancers.

The present invention is directed toward overcoming one or more of the problems discussed above.

### SUMMARY OF THE INVENTION

The present invention provides compositions and methods for treatment of cancer, and other diseases of uncontrolled cell proliferation, in patients in need thereof.

In one embodiment, the present invention contemplates t-RNA inhibitors that limit or eliminate the capacity of t-RNA to participate in translation of mRNA.

In another embodiment, the present invention contemplates 28s rRNA inhibitors that limit or eliminate the capacity of 28s rRNA from participating in ribosome complex assembly (and thereby translation of mRNA).

In another embodiment, the present invention contemplates non-functional amino acid residues for loading onto t-RNA to thereby limit or block t-RNA based translation of mRNA.

In another embodiment, the present invention contemplates compositions comprising one or more of the t-RNA inhibitors, 28s rRNA inhibitors and non-functional amino acids to limit the capacity of a cell to produce proteins, especially house keeping proteins.

In another embodiment, the present invention contemplates pharmaceutical compositions of one or more of the t-RNA inhibitors, 28s rRNA inhibitors and non-functional amino acids for use in the treatment of cancer or other like disease.

In another embodiment, the present invention contemplates methods for the treatment of patients having cancer using pharmaceutical compositions having one or more of the t-RNA inhibitors, 28s rRNA inhibitors and non-functional amino acids. In one aspect the cancer is chronic lymphocytic leukemia.

These and various other features and advantages of the invention will be apparent from a reading of the following detailed description and a review of the appended claims.

### DETAILED DESCRIPTION OF THE INVENTION

The following definitions are provided to facilitate understanding of certain terms used frequently herein and are not meant to limit the scope of the present disclosure:

“Amino acid” refers to any of the twenty naturally occurring amino acids as well as any modified amino acid sequences. Modifications can include natural processes, such as posttranslational processing, or chemical modifications which are generally known in the art. Modifications include but are not limited to: phosphorylation, ubiquitination, acetylation, amidation, glycosylation, covalent attachment of flavin, ADP-ribosylation, cross-linking, iodination, methylation, and other like modifications.

“Genetic engineering” refers to any recombinant DNA or RNA method used to create a host cell that expresses a target protein at elevated levels, at lowered levels, or in a mutated form. In general, the host cell has been transfected, transformed or transduced with a recombinant polynucleotide molecule, and thereby altered so as to cause the host cell to alter expression of the desired or target protein or peptide. Methods and vectors for genetically engineering host cells are well known in the art; for example various techniques are illustrated in Current Protocols in Molecular Biology, Ausubel et al. eds. (Wiley & Sons, New York, 1988, and quarterly updates). Genetically engineering techniques include but are not limited to expression vectors, targeted homologous recombination and gene activation (see for example, U.S. Pat. No. 5,272,071) and trans-activation by engineered transcription factors (see for example, Segal et al., 1999 Proc. Natl. Acad Sci USA 96(6):2758-63).

“Gene or nucleic acid delivery techniques” refers to the delivery of genes or nucleic acids to a specific set of cells, for example, delivery of nucleic acids to CLL cells in a patient with CLL. Methods for gene delivery include the use of ligand-associated delivery vectors. In general, vectors recognize and bind to cell surface receptors that are at least partially unique to the target cells and that may undergo endocytosis upon binding to the ligands. Receptor-vector complexes, together with membrane, can become intracellular transport vesicles. A variety of receptor-mediated intracellular gene delivery methods are known including integrin-binding proteins (Berkner, 1988 Biotechniques 6: 616-629); transferring (Zenke et al., 1990 PNAS USA 87:3655-3659); galactose (Remy et al., 1995 PNAS USA 92:1744); fibroblast growth factor (Goldman et al., 1997 Cancer 57:1447-1451); and epidermal growth factor (Schaffer et al., 1998 J Bio Chem 273:28004-009). Each of these references is incorporated by reference in their entirety. Note also that gene delivery technique also include phage delivery systems.

“Host cell(s)” refers to any cells expressing a heterologous polynucleotide molecule (for example a peptide molecule of the present invention). Example host cells of the present disclosure include, but are not limited to, insect, yeast, bacterial and mammalian cells. Specific examples of such cells include SF9 insect cells (Summers and Smith, 1987, Texas Agriculture Experiment Station Bulletin, 1555), human embryonic kidney cells (293 cells), E. coli cells, and other like cells.

“Nucleic acid (NA) sequence” refers to the order or sequence of nucleotides along a strand of polynucleotides. The order of the nucleotides can ultimately determine the order of amino acids along a polypeptide chain. Nucleotides may be ribonucleotides, deoxyribonucleotides or a mixture of both. Further, nucleotides can be modified to, for example, limit degradation by enzymatic action.

“Protein,” “peptide,” and “polypeptide” are used interchangeably herein to denote an amino acid polymer or set of two or more interacting or bound amino acid polymers.

“Treat” or “treatment” refers to remediation of the causes and/or symptoms associated with tumor and/or malignant growth, while “inhibition” refers to limiting the course of tumor and/or malignant growth as compared to the anticipated course of the disease for that particular patient.

Various embodiments of the present invention provide cell growth inhibitors that target conserved portions of t-RNA or 28s rRNA molecules. Inhibitors include polynucleotide strands that recognize and bind to the 5′ end of target tRNA molecules, and polynucleotide strands that recognize and bind to conserved regions of the 28s rRNA molecule.

In one embodiment, the formation of the double stranded RNA molecules (inhibitor+t-RNA or inhibitor+28s rRNA) initiates the RNAi pathway. For example, the double stranded RNA molecules activate the ribonuclease protein dicer which binds and cleaves the molecules into 20-25 base pair fragments (siRNAs) (see for example: Macrae et al., (2006) Science 311 (5758); Zamore et al., (2000) Cell 101(1):25-33; and Okamura et al., (2004) Genes Dev 18(14): 1655-66, each of which is incorporated by reference in its entirety for all purposes). Activation of RNAi in inhibitor treated cells leads to loss of either t-RNA or 28s rRNA and thereby limitation of protein production. This is particularly important in cells attempting to grow and divide where adequate levels of various house keeping proteins is essential, i.e., cancerous cells.

Embodiments of the present invention also provide co-administration of non-functional amino acids for loading onto t-RNA. Non-functional amino acid molecules inhibit t-RNA activity, and in combination with targeted removal of t-RNA molecules via oligonucleotide inhibitors, provides a substantial shut down of target cell translation, i.e., protein production, and ultimately cell death.

t-RNA Inhibitors

Embodiments of the invention comprise compositions for the inhibition of t-RNA activity in target cells. Complementary strands (typically single stranded) directed at conserved regions of mammalian, and in particular human, t-RNA are designed and utilized to link up to the 5 prime (5′) end of t-RNA molecules. Interaction between the t-RNA inhibitor and t-RNA blocks t-RNA activation for reading mRNA on ribosomes.

RNAi pathways are ultimately activated in cells having the t-RNA inhibitors (due to double-stranded RNA presence) rendering t-RNA molecules cleaved by RNAi processes (see for example Bantounas et al., J or Mol. Endo. (2004) 33, 545-557, incorporated by reference herein for all purposes).

In one embodiment, the tRNA inhibitor has a nucleotide sequence 5′ UGGUGCAA 3′ (SEQ ID NO:1). In another embodiment, the tRNA inhibitor has a nucleotide sequence 3′ CGCCU 5′ (SEQ ID NO:2). In yet another embodiment the tRNA inhibitor has a nucleotide sequence 3′ GAUUU 5′ (SEQ ID NO:3). And in yet another embodiment the tRNA inhibitor has a nucleotide sequence 5′ CUAAA 3′ (SEQ ID NO:4). In still another embodiment the tRNA inhibitor has a nucleotide sequence 5′ TGGCNNAGTGG 3′ (SEQ ID NO:5). Finally, in another embodiment the tRNA inhibitor has a nucleotide sequence 5′ GGTTCGANNCC 3′ (SEQ ID NO:6). Note that N sequence can represent U, T, A, G or C.

Ribosomal Inhibitors

Embodiments of the invention provide inhibitors of 28s rRNA of the large subunit of the ribosome complex. Sequences of the 28s rRNA are quite conserved among particular cell types in eukaryotes. RNAi platform technologies are employed in synthesizing complementary 28s rRNA strands that will block the assembly of the ribosome complex.

Ribosomal target sequences herein for 26-28s ribosomal rRNA is approximately 5.1 kb in length and approximately 51% conserved. (see Gonzalez et al., PNAS USA 1985 November; 82(22): 7666-7670, incorporated herein by reference in its entirety for all purposes, and McCallum and Maden, Biochem 3. 1985 Dec. 15; 232(3): 725-733, incorporated by reference herein for all purposes). Embodiments herein include rRNA inhibitors having a nucleotide sequence of: 5′ UUUUTATAIJUU 3′ (SEQ ID NO:7), 5′ TATAUUUCGCG 3′ (SEQ ID NO:8), and 5′ UUUCGCCCTATA 3′ (SEQ ID NO:9).

Non-Functional Amino Acids

Embodiments of the invention provide non-functional amino acids for loading onto t-RNA in cancer cells. Non-functional amino acids are as described previously, for example: Vaughan et al., 2005 Med. Chem. 1(3):227-37; Ataide et al., 2006 ACS Chem. Biol. 1(5)285-97; Ahel et al., 2005 FEBS Lett. 15; 579(20):4344-8; and Sando et al., 2005 J Am Chem. Soc. 127(22):7998-9, each of which is incorporated by reference herein in its entirety. As discussed more fully below, non-functional amino acids are genetically engineered for gene delivery into cancer cells to limit and/or block protein production in cancer cells.

In some embodiments the non-functional amino acids are prepared using nucleic acid analogs, including: GNA (glycerol nucleic acid); LNA (locked amino acid); PNA (peptide amino acid); TNA (threos nucleic acid) and morpholino amino acids. Target amino acids for preparation of non-functional amino acids would be prepared using the above mentioned nucleic acid analogs resulting in the non-functional amino acids of the invention.

Pharmaceutical Compositions:

Embodiments of the invention provide pharmaceutical compositions containing a substantially purified or isolated gene delivery vehicle (for production of the inhibitor of the invention in target cells) and a pharmaceutically acceptable carrier. Such pharmaceutical compositions are administered to patients in need thereof. Pharmaceutical compositions can also include chemotherapeutic drugs, antibodies, saline, anti-inflammatory drugs, and other useful medicaments for the treatment of cancer. The particular chemotherapeutic regiments for combination with the inhibitors of the invention are dependent on the type of cancer, the condition of the patient, and the standards of the patient's health care professional.

Treatment of Cancer

Embodiments of the invention can be used to treat various cancer types including breast cancer, prostate cancer, lung cancer, lymphoma, chronic lymphocytic leukemia, etc in a patient in need thereof. For purposes herein, a patient is a mammal, and more typically a human, having a cancer type in need of treatment via one or more embodiments described herein. Compositions of the invention are used to block or inhibit t-RNA and/or ribosomal RNA complex assembly in target tumor cells. Inhibition of these aspects of cell translation will quickly diminish a tumor cell's ability to produce proteins required for cellular maintenance thereby leading to cell death (for example block production of beta 2 microglobin).

Targeted delivery of t-RNA and 28s rRNA inhibitors and non-functional amino acids to tumor cells can be accomplished in a variety of manners. Typical embodiments use specific tumor cell antigens as target sites for delivery of the inhibitors of the invention. One approach is to use a phage delivery system or other known gene delivery techniques targeted at tumor cell antigens. Direct contact, inhalation, injection and other known pharmaceutical composition delivery routes may be used to administer the material to a patient in need thereof.

In one embodiment, compositions and methods of the invention are used to treat chronic lymphocytic leukemia (CLL), a type of cancer in which lymphocytes in the bone marrow of an effected patient over-proliferate in an uncontrolled manner. Nucleotide sequence for various non-functional amino acid molecules is integrated into an adenovirus phage capsule. The nucleic acid for the non-functional amino acid containing adenovirus phage is placed into a target bacterial vector for production of non-functional amino acids that are packaged into phage capsules. The phage arms have previously been identified and manipulated to recognize the CD-20 (information for this procedure is available via the VL and HL from NFCR Center for therapeutic antibody engineering, incorporated by reference herein in its entirety) marker found on the cell surface of CLL cells. The phages then will deliver the coding material for the non-functional amino acids into the CLL cells and thereby ultimately shut down protein production in the CLL cells.

In alternative embodiments, t-RNA and 28s rRNA inhibitors and non-functional amino acids of the invention are formulated as pharmaceutical compositions and administered to patients in need thereof. The inhibitors and/or non-functional amino acids can be administered in combination with pharmaceutically acceptable carriers and may be combined with specific delivery agents, including phages, antibodies or other designed molecules targeted to tumor cells.

In some embodiments the tRNA inhibitor is an inhibitor having a sequence of SEQ ID NO: 1, 2, 3, 4, 5 and/or 6. Note that one or more of the nucleic acid sequences of SEQ ID NOs: 1, 2, 3, 4, 5 or 6 can be used in a single pharmaceutical formulation. In addition, these tRNA inhibitors can be combined with one or more 28 rRNA inhibitor and/or one or more non-functional amino acid.

In some embodiments the ribosomal RNA inhibitor is an inhibitor having a sequence of SEQ ID NO: 7, 8 and/or 9. Note that one or more of the nucleic acid sequences of SEQ ID NOs: 7, 8, or 9 can be used in a single pharmaceutical formulation. In addition, these ribosomal RNA inhibitors can be combined with one or more tRNA inhibitor and/or one or more non-functional amino acid.

Inhibitors and non-functional amino acids can be administered by known techniques, such as parentally (including subcutaneous injection, intramuscular, intrasternal or infusion techniques), by inhalation spray, topically, by adsorption through a mucous membrane or rectally, in dosage unit formulations conventional to non-toxic pharmaceutically acceptable carriers, adjuvants or vehicles.

For administration by inhalation or aerosol, the compositions can be prepared according to techniques well-known in the art of pharmaceutical formulations. The compositions can be prepared as solutions in saline, using benzyl alcohol or other suitable preservatives, absorption promoters to enhance bioavailability, fluorocarbons or other solubilizing or dispersing agents known in the art.

For administration as injectable solutions or suspensions, the compositions can be formulated according to techniques well-known in the art, using suitable dispersing or wetting and suspending agents, such as sterile oils, including synthetic mono- or diglycerides, and fatty acids, including oleic acid.

In one embodiment, the inhibitor and/or non-functional amino acid compositions of the invention are administered directly to a tumor by tumor injection or by systemic delivery by intravenous injection.

Solutions or suspensions of the inhibitors and/or non-functional amino acids can be prepared in water, isotonic saline (PBS) and optionally mixed with nontoxic surfactant. Dispersions may also be prepared in glycerol, liquid polyethylene, glycols, vegetable oils, triacetin and mixtures thereof. Under normal conditions of storage and use, these preparations may contain a preservative to prevent the growth of microorganisms.

The pharmaceutical dosage form suitable for injection or infusion can include sterile, aqueous solutions or dispersions or sterile powders comprising inhibitors and/or non-functional amino acids which are adapted for the extemporaneous preparation of sterile injectable or infusible solutions or dispersions. The ultimate dosage form should be sterile, fluid and stable under the conditions of manufacture and storage. In some cases this may require that the compositions described herein be filter sterilized.

Where appropriate dosage amounts and acceptable delivery intervals are as known in the art for other like nucleic acid or amino acid based therapeutics, e.g., aptamers.

Within the application, unless otherwise stated, the techniques utilized may be found in any of several well-known references, such as: Molecular Cloning: A Laboratory Manual (Sambrook et al. 1989 Molecular cloning: A Laboratory Manual), Gene Expression Technology (Methods in Enzymology, V 185, edited by D. Goeddel, 1991 Academic Press, San Diego, Calif.), PCR Protocols: A Guide to Methods and Applications (Innis et al. (1990) Academic Press, San Diego, Calif.), and Gene Transfer and Expression Protocols, pp 109-128, ed EJ Murray, The Humana Press Inc., Clifton, N.J.).

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims.

1. A method for the treatment of cancer in a patient in need thereof comprising:
nucleic acid delivery of one or more t-RNA inhibitors to tumor cells in the patient in need thereof wherein the t-RNA inhibitor limits protein production in tumor cells and thereby causes the tumor cells to preferentially die as compared to other non-tumor cells in the patient.

2. A method for the treatment of cancer in a patient in need thereof comprising:
nucleic acid delivery of one or more 28s rRNA inhibitors to tumor cells in the patient in need thereof wherein the 28s rRNA inhibitor limits protein production in tumor cells and thereby causes the tumor cells to preferentially die as compared to other non-tumor cells in the patient.

3. The method of claim 1 further comprising nucleic acid delivery of non-functional amino acid molecules for inhibition of t-RNA activity wherein the non-functional amino acid molecules further limit protein production in tumor cells and thereby further cause tumor cells to preferentially die as compared to non-tumor cells in the patient.

4. The method of claim 2 further comprising nucleic acid delivery of non-functional amino acid molecules for inhibition of t-RNA activity wherein the non-functional amino acid molecules further limit protein production in tumor cells and thereby further cause tumor cells to preferentially die as compared to non-tumor cells in the patient.

5. The method of claim 1 wherein the cancer is CLL.

6. The method of claim 2 wherein the cancer is CLL.

7. The method of claim 5 wherein the nucleic acid delivery is through the use of a phage delivery system.

8. The method of claim 6 wherein the nucleic acid delivery is through the use of a phage delivery system.

9. The method of claim 7 wherein the phage delivery system uses the CD-20 cell surface marker to specifically target tumor cells for delivery of the appropriate inhibitor.

10. The method of claim 8 wherein the phage delivery system uses the CD-20 cell surface marker to specifically target tumor cells for delivery of the appropriate inhibitor.

11. The method of claim 1 wherein the one or more tRNA inhibitor has a sequence selected from the group consisting of: SEQ ID NO: 1, SEQ ID NO: 2, SEQ ID NO: 3 and SEQ ID NO: 4.

12. The method of claim 11 wherein the one or more tRNA inhibitor is two tRNA inhibitors selected from the group consisting of: SEQ ID NO: 1, SEQ ID NO: 2, SEQ ID NO: 3 and SEQ ID NO: 4.

13. An isolated nucleic acid having a sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8.

14. The isolated nucleic acid of claim 13, having a sequence of SEQ ID NO: 2.

15. The isolated nucleic acid of claim 13, having a sequence of SEQ ID NO: 3.

16. The isolated nucleic acid of claim 13, having a sequence of SEQ ID NO: 4.

17. The isolated nucleic acid of claim 13, having a sequence of SEQ ID NO: 5.

18. The isolated nucleic acid of claim 13, having a sequence of SEQ ID NO: 6.

19. The isolated nucleic acid of claim 13, having a sequence of SEQ ID NO: 7.

20. The isolated nucleic acid of claim 13, having a sequence of SEQ ID NO: 8.
